(19) United States
(12) Patent Application Publication
(10) Pub. No.: US 2010/0125286 A1
(43) Pub. Date: May 20, 2010

(54) Surgical Positioning Device and Image Guided Navigation System Using The Same

(76) Inventors: Jaw-Lin Wang, Taipei City (TW); Yao-Hung Wang, Taipei City (TW); Been-Der Yang, Taipei City (TW); Chi-Lin Yang, Taipei City (TW)

Correspondence Address: KAMRATH & ASSOCIATES P.A., 4825 OLSON MEMORIAL HIGHWAY, SUITE 245, GOLDEN VALLEY, MN, 55422, US

(21) Appl. No.: 12/621,577

(22) Filed: Nov. 19, 2009

(30) Foreign Application Priority Data
Nov. 20, 2008 (TW) 097144906

(51) Int. Cl. A61B 19/00

(52) U.S. Cl. 606/130

(57) ABSTRACT

A surgical positioning device comprises a base, an upper moving structure, a lower moving structure, an auxiliary operating element, and a plurality of encoders. The upper moving structure and the lower moving structure are coupled to the base individually, such that the upper moving structure and lower moving structure may move on a first plane and a second plane, respectively. The first plane is substantially parallel to the second plane. Each of the upper moving structure and lower moving structure comprise a holder respectively for holding the auxiliary operating element. The plurality of encoders measure the movement of the moving elements of the upper moving structure and the lower moving structure. A surgical tool may be adjusted and positioned by operating the auxiliary operating element to move the upper moving structure and the lower moving structure on the different planes, respectively.

### BACKGROUND

The invention relates to a surgical positioning device; in particular, the invention relates to a microminiaturized surgical positioning device for real time tracking and positioning surgical tools during surgery.

One common process in surgery is to perform needle placement under image guidance. For example, some common applications are pedicle screw insertion, biopsy, and spinal epidural blocks. The direction of movement and the final position of a needle are guided during surgery by constant monitoring of fluoroscopic images (dynamic X-ray). Accurate needle placement may provide guided routes for subsequent surgical tools to achieve effective treatment. For example, in pedicle screw insertion, a surgeon may first place a guiding needle near the treatment site of the spine to establish surgery routes from the skin to the treated vertebra. Then a tiny opening is made in the skin to place the pedicle screws into the vertebra along the needle routes. The abovementioned surgery, which has the advantages of small incision, low infection risk, and rapid recovery, is generally called Minimally Invasive Surgery. However, because the position and the direction of a needle in the body cannot be observed with the eyes, accurate needle placement is a very difficult task. It is necessary to constantly monitor fluoroscopic images during surgery to observe and then adjust the direction of movement and the final position of a needle. However, guiding the direction of movement of a needle in space according to planar fluoroscopic images requires highly experience. Especially, with concern for the life of the patient, the difficulty of this task causes a heavy burden for some less-experienced surgeons. Furthermore, repeated excessive exposures to X-rays during such processes can have serious consequences for the health of medical personnel.

With advancements in computers and medical imaging technology, navigation systems for computer-aided surgery are utilized to provide safe and effective needle placement for surgeons. An image guided navigation system utilizes CT (computerized axial tomography) scan images of a patient acquired before surgery as guidance media to display real-time needle images in a display system through trackers, without checking the position of the needle with fluoroscopic images. Thus, surgeons can perform minimally invasive surgery in an environment free of irradiation. Furthermore, compared to overlapping fluoroscopic images, CT scan images can provide images for surgeons to identify anatomical structure surrounding the needle more accurately to achieve surgery results with high accuracy. The important issues for surgeons during needle placement are to determine needle routes with the least harm and to avoid possible neurovascular injury. Thus, first, for the operation of the navigation system, a possible entrance point is selected on the skin by a tracker of the navigation system. Then the directions of the tracker are swung around the entrance point as a center. With the CT scan image system, the direction and the position of the target point can be found. Subsequently, the planned route can be evaluated to ensure that no vital organs will be punctured. If the needle route is not suitable, the entrance point can be changed and the route can be evaluated again until an optimal route is found. Once the entrance point and the direction of needle are decided, the needle is placed into the target position. The abovementioned image guided navigation system is different from the conventional image guided surgery. In conventional image guided surgery the needles are adjusted inside the body in a stepwise manner, while the abovementioned navigation system simulates the needle puncture process with images acquired before surgery. Thus, the direction and the position of needle can be decided without invading the body, greatly reducing the harm caused to the patient by the needle.

Trackers are essential devices in computer-aided navigation systems. Trackers trace the real-time positions of a needle in space to create corresponding anatomical images of patients for display on an image system. To display the corresponding images correctly, it is necessary to establish corresponding relations between images before surgery and the patient during surgery. The establishment of corresponding relations is called registration. Registration is accomplished by identifying marker points in CT scan images, measuring the marker points by utilizing trackers as measurement tools, and calculating a coordinate transformation matrix with mathematical algorithms. The coordinate transformation matrix converts the position of the needle in the physical space to the position in the image system in real time to display the virtual image of the needle superimposed on the corresponding anatomical image. The patient may move during surgery, and any slight movement may change the result of registration. Thus, another important function of trackers is to maintain the registration relations between patients and trackers. This function is called tracking. With the tracking function, the image guided navigation system can track the real-time positions of needles correctly.

Common image guided navigation systems utilize optical infrared cameras as trackers. For example, the related technology is disclosed in U.S. Pat. No. 6,348,058, U.S. Pat. No. 6,947,786, and U.S. Pat. No. 6,340,363. The registration function of optical trackers is achieved by measuring artificial markers or anatomical markers of a body, such as bone landmarks, and by calculating coordinate transformation with the corresponding marker positions in CT scan images. The tracking function of optical trackers is achieved by embedding a dynamic reference frame into a body. Generally, a dynamic reference frame is equipped with three to four reflective markers and is affixed on a rigid anatomy of the patient, such as bone. The protruding reference points moved with the patient are tracked accordingly. This method is time consuming because of extra pre-operative preparation and intro-operative measurement. It also causes additional injury to patients. Moreover, the valid range of an optical tracker relates to the distance from the system to the reference point. To track reference points within surgery range, the trackers must be placed away the surgery area distantly. As a result, the system may fail if the sight of the reference point is blocked.

Another type of the navigation system utilizes articulate mechanical arms as trackers. The related technology is disclosed in U.S. Pat. No. 5,230,623. Such a position measurement device adopts serial link structures to avoid the tracking failure problem inherent to the optical navigation system. But they cannot track the positions of patients constantly. The mentioned position measurement devices must update positions by repeatedly measuring marker points or immobilize the patient to the surgery table to prevent any inadvertent movement in order to maintain position accuracy. This kind of serial link device occupies the already crowded operation room because of inevitable huge volume. Furthermore, any movement of the needle, even a small shift, involves the position changes of all links, making that a delicate action is difficult to perform.

### SUMMARY

The main object of the present invention is to provide a design with two parallel moving mechanisms to adjust and position a surgical tool easily.

To achieve the above object, a surgical positioning device for positioning a surgical tool of the present invention comprises a base, an upper moving structure, a lower moving structure, an auxiliary operating element, and a plurality of encoders. The base provides image registration functions. The upper moving structure comprises a first moving element, a second moving element, and an upper holder. The first moving element is coupled to the base. One end of the second moving element is coupled to the first moving element, and the other end of the second moving element is connected to the upper holder, to allow the upper moving structure to move arbitrarily on a first plane. The lower moving structure comprises a third moving element, a fourth moving element, and a lower holder. The third moving element is coupled to the base. One end of the fourth moving element is coupled to the third moving element, and the other end of the fourth moving element is connected to the lower holder, to allow the lower moving structure to move arbitrarily on a second plane. The auxiliary operating element is held by the upper holder and the lower holder. The plurality of encoders measure movements of each of the moving elements, wherein the upper moving structure and the lower moving structure are capable of moving on different planes respectively by operation of the auxiliary operating element to adjust and position the surgical tool. By the design of the present invention, the volume of the surgical positioning device can be reduced. Thus, the surgical positioning device of the present invention can be placed directly on the patient's body to compensate for possible movements of the patient during surgery to achieve tracking positions of surgical tools in real time.

The present invention also provides an image guided navigation system comprising a computer and the abovementioned surgical positioning device. The surgical positioning device positions a surgical tool. The computer system comprises a storage medium, a processor, and a display. The storage medium stores a plurality of CT scan images. The processor, electrically coupled to the storage medium and the plurality of encoders, processes movements of the moving elements and acquires information on the position of the surgical tool by utilizing the image registration functions of the base. A corresponding image is obtained from the plurality of CT scan images according to the position information. The display, electrically coupled to the processor, shows the corresponding image. Thus, when surgeons use the image-guided navigation systems of the present invention, the steps of measuring marker points and identifying marker positions in images are not necessary. Tracking of the positions of surgical tools can be automatically achieved and surgery performance can be improved.

### DESCRIPTION OF THE DRAWINGS

The invention can be more fully understood by reading the subsequent detailed description and examples with references made to the accompanying drawings, wherein:

FIG. 1 is a diagram of a structure of a surgical positioning device of the present invention.

FIG. 2 is a diagram of actions of a surgical positioning device of the present invention.

FIG. 3 is a diagram of utilization states of a surgical positioning device of the present invention.

FIG. 4 is a diagram of another embodiment of a base of a surgical positioning device of the present invention.

FIG. 5 is a diagram of an image guided navigation system of the present invention.

### DESCRIPTION

The invention can be more fully understood by reading the subsequent detailed description and examples with references made to the accompanying drawings.

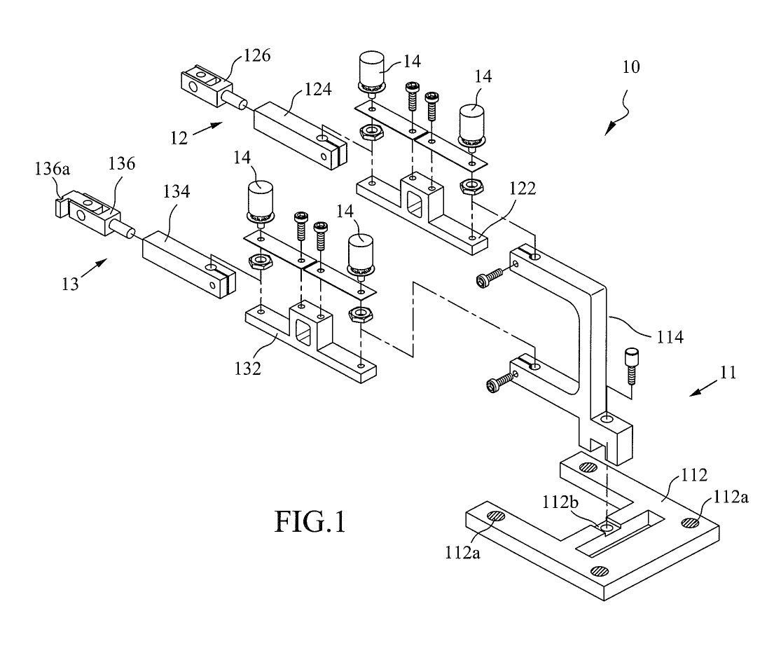

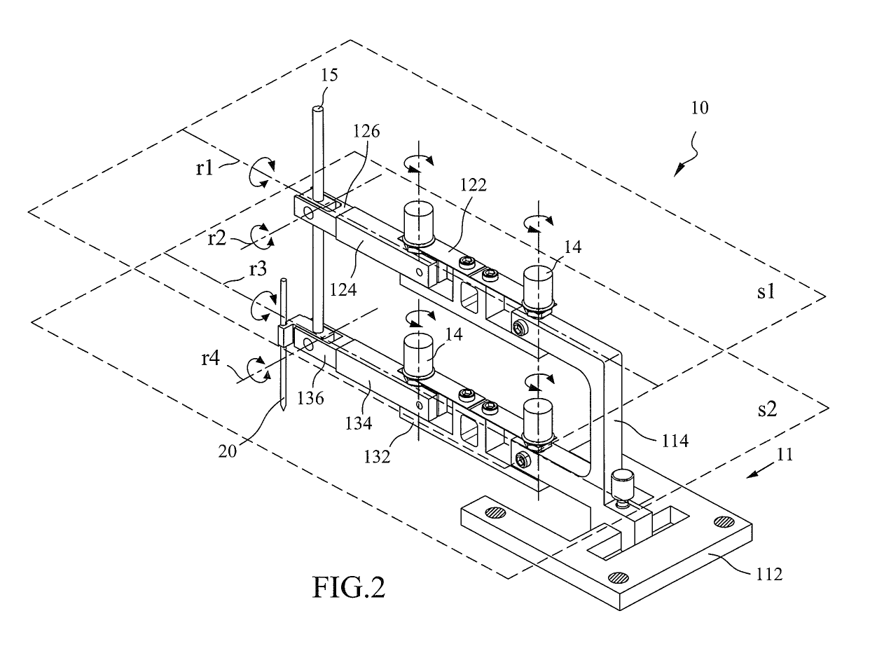

Referring to FIG. 1 and FIG. 2, FIG. 1 is a diagram of a structure of a surgical positioning device 10 of the present invention. FIG. 2 is a diagram of actions of a surgical positioning device 10 of the present invention. The surgical positioning device 10 of the present invention positions a surgical tool 20, as shown in FIG. 1. The surgical positioning device 10 of the present invention comprises a base 11, an upper moving structure 12, a lower moving structure 13, an auxiliary operating element 15, and a plurality of encoders 14. The base 11 comprises a pedestal 112 and a stand 114, and the pedestal 112 can be combined with the stand 114. The pedestal 112 comprises a plurality of positioning marker elements 112a to provide image registration functions. The pedestal 112 further comprises at least one positioning slot 112b. The surgeons can adjust surgery range by inserting the stand 114 correspondingly into the positioning slots 112b selectively.

With the mentioned image registration functions, surgeons can mount the pedestal 112 of the surgical positioning device 10 of the present invention on a patient's body, and then acquire CT scan images of the patient. During the acquisition of the CT scan images of a patient, because the plurality of positioning marker elements 112a are installed in the pedestal 112 of the base 11, the positioning marker elements 112a appear in the CT scan images as registration markers. The coordinates and relative positions of the positioning marker elements 112a can be established through image analysis and image processing of the CT scan images by computers or other processing devices. Thus, the position locations and relative information can be confirmed to achieve the purposes of automatic registration. The above computer processing technology of the image registration has been disclosed in related patents and documents, and the details are not described herein. Then the stand 114 can be combined with the pedestal 112. The coordinates of the positioning slots 112b of the pedestal 112 are known in advance. The processing device calculates the coordinates of a surgical tool according to the known specifications of the pedestal 114 and other moving structures, and the relative moving information obtained from other moving structures. Therefore, the goal of tracking positions of a surgical tool can be achieved.

The upper moving structure 12 comprises a first moving element 122, a second moving element 124, and an upper holder 126. The first moving element 122 is coupled to the stand 114 of the base 11. One end of the second moving element 124 is coupled to the first moving element 122, and the other end of the second moving element 124 is connected to the upper holder 126 to form the upper moving structure 12 as a linked moving assembly. As shown in FIG. 2, the axis on which the first moving element 122 and the stand 114 are coupled is substantially parallel to the axis on which the second moving element 124 and the first moving element 122 are coupled. Thus, the upper moving structure 12 can be moved arbitrarily on a first plane s1.

Similarly, the lower moving structure 13 comprises a third moving element 132, a fourth moving element 134, and a lower holder 136. The third moving element 132 is coupled to the stand 114 of the base 11. One end of the fourth moving element 134 is coupled to the third moving element 132, and the other end of the fourth moving element 134 is connected to the lower holder 136 to form the lower moving structure 13 as a linked moving assembly. As shown in FIG. 2, the axis on which the third moving element 132 and the stand 114 are coupled is substantially parallel to the axis on which the fourth moving element 134 and the third moving element 132 are coupled. Thus, the lower moving structure 13 can be moved arbitrarily on a second plane s2.

The first moving element 122 and the third moving element 132 are coupled to the stand 114 in the same axis to form the upper moving structure 12 and the lower moving structure 13 as a parallel moving structure. The first plane s1 is substantially parallel to the second plane s2, and the upper holder 126 and the lower holder 136 hold the auxiliary operating element 15. In this embodiment, the lower holder 136 comprises a holding structure 136a for installing and fixing the surgical tool 20. However, the present invention is not limited by the above designs and embodiments. The surgical positioning device 10 may be combined with other structural designs or other surgical tools 20, such as the auxiliary operating elements 15 is a hollow pipe to install and fix the surgical tool 20 in. Furthermore, the surgical tool 20 may be a needle, a knife, a drill, or any other type of surgical tool, according to the needs of surgeons.

As shown in FIG. 2, the upper holder 126 can rotate around a first axis r1 and a second axis r2. The first axis r1 is substantially perpendicular to the second axis r2, and the first axis r1 is located on the first plane s1. The lower holder 136 can rotate around a third axis r3 and a fourth axis r4. The third axis r3 is substantially perpendicular to the fourth axis r4, and the third axis r3 is located on the second plane s2. During operation of the auxiliary operating element 15 to shift the upper moving structure 12 and the lower moving structure 13, the upper holder 126 and the lower holder 136 may not remain on the line which is substantially perpendicular to the first plane s1 and the second plane s2. The design of the present invention can allow the upper holder 126 and the lower holder 136 to rotate according to the different placement positions and also drive the surgical tool 20 of the surgical positioning device 10 to change its direction and position. Thus, surgeons may hold the auxiliary operating element 15 for operating during surgery. The lower moving structure 13 is shifted to drive the lower holder 136 to control the position of the surgical tool 20. The upper moving structure 12 is shifted to drive the upper holder 126 to control the direction of the surgical tool 20. Thus, the surgical positioning device 10 of the present invention can form an operating structure with four degrees of freedom (constituted by two translations and two rotations) to achieve the functions of adjusting flexibly the surgical tool 20. In this embodiment, the upper holder 126 or the lower holder 136 can be a gimble-type linked structure or other structures with the same function, such as spherical bearings.

The plurality of encoders 14 are installed in the couplings of the first moving element 122 and the base 11, the first moving element 122 and the second moving element 124, the third moving element 132 and the base 11, and the third moving element 132 and the fourth moving element 134. The plurality of encoders 14 measure the movement of the first moving element 122 and the third moving element 132 in relation to the base 11, the movement of the second moving element 124 in relation to the first moving element 122, and the movement of the fourth moving element 134 in relation to the third moving element 132.

When the upper moving structure 12 is shifted on the first plane s1, the movements of the first moving element 122 and the second moving element 124 are measured to define the pointing direction of the surgical tool 20. Also, when the lower moving structure 13 is shifted on the second plane s2, the movements of the third moving element 132 and the fourth moving element 134 are measured to define the position of the surgical tool 20. Thus, the positioning function of the surgical tool 20 is provided.

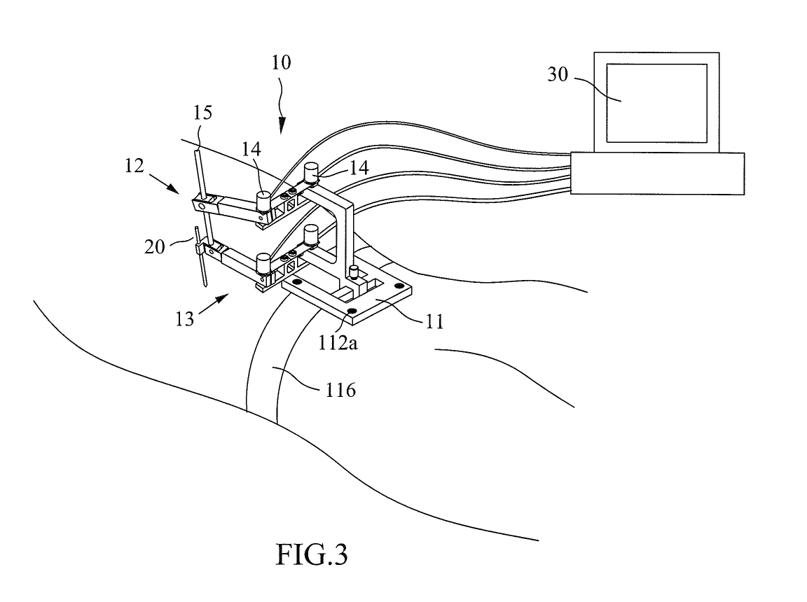

Please refer to FIG. 3. FIG. 3 is a diagram of utilization states of a surgical positioning device of the present invention. As shown in FIG. 3, the surgical positioning device 10 of the present invention can be microminiaturized for placement onto the body of a patient for surgery, so as to compensate for possible movements of the patient during surgery. The base 11 of the surgical positioning device 10 may comprise a holding element 116. The holding element 116 holds the surgical positioning device 10 to the body of a patient. In this embodiment, the holding element 116 can be, but is not limited to, a buckled belt module or other holding elements with holding functions.

The plurality of encoders 14 of the surgical positioning device 10 of the present invention may connect to a computer system 30. By utilizing the surgical positioning device 10 as a tracker, when surgeons hold the auxiliary operating element 15 for operating, the movements of each moving element of the upper moving structure 12 and the lower moving element 13 can be acquired through the plurality of encoders 14. The computer system 30 processes the movement of each moving element and coordinates with the image registration function of the base 11 to obtain the position information of the surgical tool 20. The position information includes the direction and position of the surgical tool 20. The image guided navigation system may display corresponding images of the surgical tool 20, pointing to a position of a body of a patient, for surgeons to make decisions during surgery. Using a needle as an example, the entrance point of a needle is adjusted by the lower moving structure 13, and the entrance direction of the needle is adjusted by the upper moving structure 12. The image guided navigation system displays corresponding images of the needle position and direction according to the position information returned by the surgical positioning device 10 of the present invention. Then, from the images, surgeons can observe the surrounding anatomy of the targeting needle route and determine if the needle route is suitable.

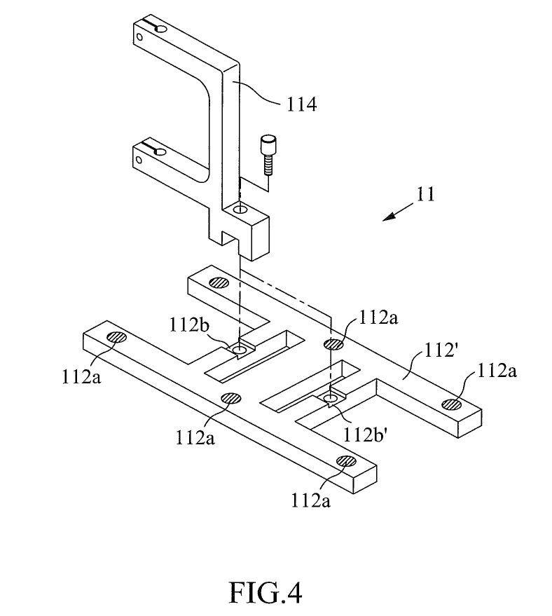

Please refer to FIG. 4. FIG. 4 is a diagram of another embodiment of a base 11 of a surgical positioning device 10 of the present invention. As shown in FIG. 4, in this embodiment, the pedestal 112′ of the base 11 includes two positioning slots 112b and 112b′, installed individually in different parts of the pedestal 112′. When the stand 114 is combined with one positioning slot 112b of the pedestal 112′, the surgical positioning device 10 can be operated only in a certain surgery range because of structural limitations. To avoid replacement and repositioning of the surgical positioning device 10 when operating the surgical positioning device 10 on different locations in a certain area during the surgery, surgeons can choose to insert the stand 114 correspondingly into any positioning slot 112b or 112b′, thereby expanding the surgery range. Also, other positioning slots may be added to the pedestal 112′ according to requirements to provide the convenience in surgery. Because the plurality of positioning marker elements 112a are installed around each positioning slot 112b or 112b′, the positioning slots 112b or 112b′ are automatically registered by the computer system during the mentioned CT scan image acquisition process. Likewise, the plurality of pedestals 112′ can be installed on different surgery locations. Thus, the surgery range can be expanded by combining the stand 114 and connecting moving structures to the different pedestals 112′ during surgery.

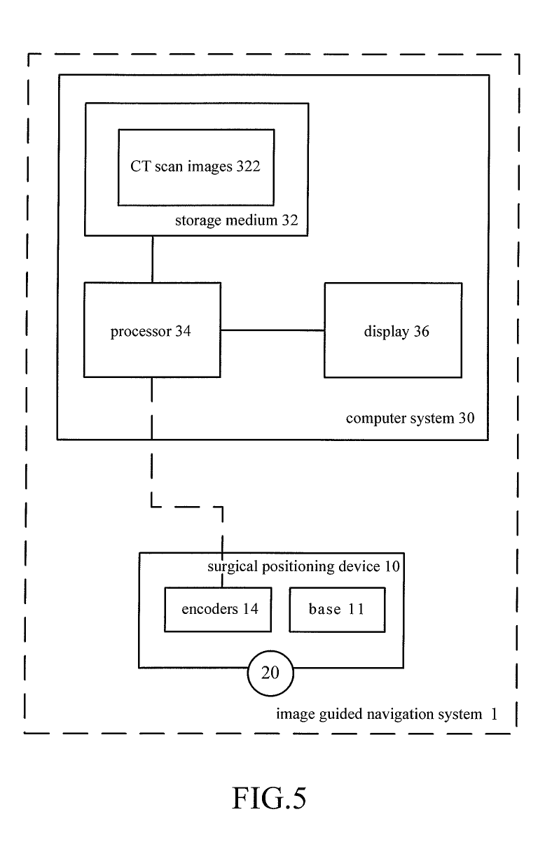

Please refer to FIG. 5. FIG. 5 is a diagram of an image guided navigation system of the present invention. The image guided navigation system 1 is utilized to track a surgical tool 20 during the surgery and to acquire CT scan images of the surgery area of a patient before the surgery. The image guided navigation system 1 of the present invention includes a computer system 30 and the mentioned surgical positioning device 10. The surgical positioning device 10 positions a surgical tool 20. The computer system 30 comprises a storage medium 32, a processor 34, and a display 36. The storage medium stores the CT scan images 322. The processor 34 is coupled to the storage medium 32 and the plurality of encoders 14 electronically. The processor 34 processes the movements of the moving elements and acquires position information of the surgical tool 20 by utilizing the image registration functions of the base 11. Then the processor 34 can obtain at least one corresponding image from the plurality of CT scan images 322 according to the position information. The display 36, electrically coupled to the processor 34, displays the at least one corresponding image. Surgeons may acquire CT scan images after the pedestal 112 of the surgical positioning device 10 is mounted on the body of a patient. Then the positioning marker elements 112a installed in the pedestal 112 can appear in the CT scan images as registration markers. The acquired images are processed by the processor 34 to establish relative positions and coordinates of the plurality of positioning marker elements 112a. Thus, the position and the relative information of the pedestal 112 are confirmed to achieve the function of automatic registration. The stand 114 and other moving structure are combined with the pedestal 112, the coordinates of which are known. The computer calculates the known coordinates according to pedestal 114 and known specifications of other moving structures and relative moving information obtained from other moving structures to achieve the object of tracking positions of a surgical tool 20. Thus, surgeons may operate directly the surgical positioning device 10 of the image guided navigation system 1 of the present invention to change the direction and position of the surgical tool 20. Also, the surgical tool 20 can be tracked in real time. The computer system 30 may display the CT scan images (including sectional images of anatomy or simulations of fluoroscopic images) of corresponding areas of the patient for confirmation of the optimal surgery needle route. The efficiency and accuracy of surgery may be upgraded.

While the invention has been described by way of example and in terms of a preferred embodiment, it is to be understood that the invention is not limited thereto. Those skilled in the technology can still make various alterations and modifications without departing from the scope and spirit of this invention. Therefore, the scope of the present invention shall be defined and protected by the following claims and their equivalents.

What is claimed is:

1. A surgical positioning device for positioning a surgical tool, comprising:
a base, providing image registration functions;
an upper moving structure, comprising a first moving element, a second moving element, and an upper holder, wherein the first moving element is coupled to the base, one end of the second moving element is coupled to the first moving element, and the other end of the second moving element is connected to the upper holder to allow the upper moving structure to move arbitrarily on a first plane;
a lower moving structure, comprising a third moving element, a fourth moving element, and a lower holder, wherein the third moving element is coupled to the base, one end of the fourth moving element is coupled to the third moving element, and the other end of the fourth moving element is connected to the lower holder to allow the lower moving structure to move arbitrarily on a second plane;
an auxiliary operating element, held by the upper holder and the lower holder; and
a plurality of encoders, measuring movement of each of the moving elements;
wherein the upper moving structure and the lower moving structure are capable of moving on different planes respectively by operation of the auxiliary operating element to adjust and position the surgical tool.

2. The surgical positioning device as claimed in claim 1, wherein the base comprises a pedestal and a stand; the pedestal can be combined with the stand, and the pedestal comprises a plurality of positioning marker elements to provide image registration functions.

3. The surgical positioning device as claimed in claim 2, wherein the pedestal further comprises at least one positioning slot for adjusting surgery range by inserting the stand correspondingly to the positioning slots selectively.

4. The surgical positioning device as claimed in claim 1, wherein the plurality of encoders are installed in the couplings of the first moving element and the base, the first moving element and the second moving element, the third moving element and the base, and the third moving element and the fourth moving element.

5. The surgical positioning device as claimed in claim 1, wherein the upper holder can rotate around a first axis and a second axis; the first axis is substantially perpendicular to the second axis, and the first axis is located on the first plane.

6. The surgical positioning device as claimed in claim 1, wherein the lower holder can rotate around a third axis and a fourth axis; the third axis is substantially perpendicular to the fourth axis, and the third axis is located on the second plane.

7. The surgical positioning device as claimed in claim 1, wherein the base further comprises a holding element providing holding functions on a body.

8. The surgical positioning device as claimed in claim 1, wherein the plurality of encoders can be connected electrically to a computer system, and the computer system processes the movement of each of the moving elements and acquires position information of the surgical tool in coordination with the image registration functions of the base.

9. The surgical positioning device as claimed in claim 8, wherein the position information comprises the direction and position of the surgical tool.

10. An image guided navigation system, tracking a surgical tool during surgery and acquiring a plurality of CT (computerized axial tomography) scan images of a surgery area of a patient in advance, comprising:
A surgical positioning device, comprising:
A base, providing image registration functions;
an upper moving structure, comprising a first moving element, a second moving element, and an upper holder, wherein the first moving element is coupled to the base, one end of the second moving element is coupled to the first moving element, and the other end of the second moving element is connected to the upper holder to allow the upper moving structure to move arbitrarily on a first plane;
a lower moving structure, comprising a third moving element, a fourth moving element, and a lower holder, wherein the third moving element is coupled to the base, one end of the fourth moving element is coupled to the third moving element, and the other end of the fourth moving element is connected to the lower holder to allow the lower moving structure to move arbitrarily on a second plane;
an auxiliary operating element, held by the upper holder and the lower holder; and
a plurality of encoders, measuring movement of each of the moving elements;
wherein the upper moving structure and the lower moving structure are capable of moving on different planes respectively by operation of the auxiliary operating element to adjust and position the surgical tool;
and
a computer system, comprising:
a storage medium, storing the plurality of CT scan images;
a processor, coupled to the storage medium and the plurality of encoders electronically, processing the movement of the moving elements, acquiring position information of the surgical tool by utilizing the image registration functions of the base, and obtaining at least one corresponding image from the plurality of CT scan images according to the position information; and
a display, coupled to the processor electronically, displaying the at least one corresponding image.

11. The image guided navigation system as claimed in claim 10, wherein the base comprises a pedestal and a stand; the pedestal can be combined with the stand, and the pedestal comprises a plurality of positioning marker elements to provide image registration functions.

12. The image guided navigation system as claimed in claim 11, wherein the pedestal further comprises at least one positioning slot for adjusting surgery range by inserting the stand correspondingly into the positioning slots selectively.

13. The image guided navigation system as claimed in claim 10, wherein the plurality of encoders are installed in the couplings of the first moving element and the base, the first moving element and the second moving element, the third moving element and the base, and the third moving element and the fourth moving element.

14. The image guided navigation system as claimed in claim 10, wherein the upper holder can rotate around a first axis and a second axis; the first axis is substantially perpendicular to the second axis, and the first axis is located on the first plane.

15. The image guided navigation system as claimed in claim 10, wherein the lower holder can rotate around a third axis and a fourth axis; the third axis is substantially perpendicular to the fourth axis, and the third axis is located on the second plane.

16. The image guided navigation system as claimed in claim 10, wherein the base further comprises a holding element providing holding functions on a body.

17. The image guided navigation system as claimed in claim 10, wherein the position information comprises the direction and position of the surgical tool.
